(19) United States
(12) Patent Application Publication
(10) Pub. No.: US 2005/0049468 A1
(43) Pub. Date: Mar. 3, 2005

(54) Increasing the performance of an optical pulsoximeter

(76) Inventors: Sven-Erik Carlson, Herrliberg (CH); Urban Schnell, Ins (CH); Deborah Schegg, Koniz (CH); Martin Liechti, Bern (CH)

Correspondence Address: NOTARO AND MICHALOS, 100 DUTCH HILL ROAD, SUITE 110, ORANGEBURG, NY, 10962-2100, US

(21) Appl. No.: 10/654,184

(22) Filed: Sep. 3, 2003

(51) Int. Cl. A61B 5/00

(52) U.S. Cl. 600/323; 600/336

(57) ABSTRACT

Proposed is a configuration for the acquisition and/or monitoring of medical data, in particular the state of the cardiovascular and pulmonary system, blood values or blood composition, characterised by at least one measuring sensor for the acquisition of the medical data such as the state of the cardiovascular system, etc. of a person comprising at least one light source which can emit light at least at two wavelengths, as well as at least one light receiver for determining the light transmitted and/or reflected through a tissue portion of a person or an animal further comprising means in order to increase the optical Signal-to-Noise and/or Signal-to-Background ratio.

The present invention refers to a configuration for the acquisition and/or monitoring of medical data according to the introduction of claim 1 and a method for the acquisition and/or monitoring of the state of health or of medical data of a person or an animal.

The invention relates in particular to optical pulsoximetry used for non-invasive measurement of pulsation and oxygen saturation in arterial human or animal blood, and is particularly concerned with increasing the technical performance of pulsoximetry in terms of quality and robustness of the measurement signal versus environmental disturbances and energy consumption.

Pulsoximetry is a widely used standard optical technology for non-invasive monitoring of pulsation and oxygen saturation in arterial human or animal blood [1]. The method consists of measuring the absorption of reduced (Hb)—and oxidized (HbO₂) haemoglobin at two optical wavelengths, where the relative absorption coefficients differ significantly, e.g. 660 nm and a second wavelength in the range of 800 to 1000 nm, preferably 890 nm or 950 nm. A concise description of the measurement method and the sensor signals is given in [2].

Commercially available pulsoximeter sensors are typically used in hospitals and doctor's offices where the (optical) environment and mounting of the sensor onto the patient's skin are well defined. In the recent past pulsoximetry measuring devices and methods are also offered and used for mobile monitoring and surveying of human individuals, e.g. suffering of diseases, such as heart problems, diabetes, respiratory diseases, insufficient oxygen blood saturation, etc. Pulsoximetry measuring devices are also used in sports for control and survey of athletes. Respective monitoring devices are described within the international patent application WO02/089 663 which proposes in this respect to monitor in particular persons with cardio vascular disorders by means of pulsoximetry with measurements being taken by means of pulsoximetry preferably on an ear or on a finger. When using pulsoximetry in telemedicine or near patient testing applications, which means e.g. at self-controlling and self-testing of patients in non-ideal environment, standard pulsoximeter sensors suffer from signal instability and insufficient robustness versus environmental disturbances.

Critical points are:
Human tissue scatters and transmits light in the visible and near infrared (NIR) wavelength range. Therefore, suppression of environmental optical radiation, e.g. sunlight, is difficult by geometric means of the architecture of the pulsoximeter sensor.
The power spectrum of environmental optical radiation strongly varies as a function or time and place where the pulsoximeter is used, e.g. day versus night, indoor versus outdoor. Therefore, the background (offset) in the detected optical power varies in a large range, making difficult the analog and digital signal processing of the primary sensor signal.
The temporal spectrum of pulsoximeter signals varies in the range of 0.5 Hz to 5 Hz where environmental optical radiation may have significant components leading to parasitic contributions which cannot be separated from the pulsoximeter signals of interest.
Realization of a performing electronic band pass filter in the range of 0.5 Hz to 5 Hz, in order to suppress DC offset and high frequency contribution in the pulsoximeter signal, is technically challenging. Further, optical contributions, e.g. temporally structured day-light, and electronic noise, e.g. 1/f (1/frequency-Noise), are stronger in the low frequency range 0.5 Hz to 10 Hz than in higher frequency ranges.

It is therefore an object of the present invention to define optical and/or electronic means for increasing the Signal-to-Noise ratio (S/N) and Signal-to-Background ratio (S/B) of a pulsoximeter sensor for robust application of pulsoximetry in telemedicine- and near patient testing applications in rough (optical) environmental conditions, e.g. at changing light influences, such as sunlight, shadow, artificial light, etc.

The posed problem is solved by means of a configuration/method according to the invention. Proposed is a configuration for monitoring which comprises at least one of the following components:
at least one measuring sensor for the acquisition of the medical data, such as the state of the cardiovascular and pulmonary system, as e.g. pulsation frequency, oxygen saturation of blood, breathing frequency, etc. of a human being or an animal, comprising at least one light source which can emit light at least at two wavelengths, as well as at least one light receiver for determining the light transmitted through a tissue portion of the person or the animal;
at least one beam shaping optical element to direct the emitted light to a human or animal tissue and the light receiver in order to increase the optical signal power.

The basic idea therefore is to use a beam-shaping element, such as e.g. diffractive or refractive lenses, to direct the emitted optical radiation of, e.g., the LED light source into the human or animal tissue and the photon detecting element in order to increase the optical signal power, detected by the pulsoximeter sensor, and thus increasing the Signal/Noise—and signal/Background ratio. The increase of the S/B ratio is estimated e.g. to a factor 5.

In addition to the above mentioned configuration or as an alternative, it is proposed to use a configuration for monitoring e.g. pulsation frequency, oxygen saturation within blood and breathing frequency which comprises at least the following components:
at least one measuring sensor to the person or the animal for the acquisition or monitoring of medically relevant data which sensor comprises at least one light source that can emit light at least at two wavelengths, as well as at least one light receiver for determining the light transmission through a tissue portion of the person or the animal, and
at least one light baffle or light trap, respectively, and/or an optical wavelength filter which is adapted to the power spectrum of the light source and the absorption spectrum of human or animal arterial blood. The basic idea of using geometric baffles or light traps, respectively, and/or optical wavelength filters is to suppress by geometric and/or optical means the parasitic contribution of environmental radiation in order to increase or stabilize the S/B (Signal/Background) ratio vs. environmental conditions. The increase of the S/B ratio is e.g. estimated to a factor 10-100.

Again, in addition to the above mentioned two configurations, or as an alternative, a further configuration is proposed which comprises at least the following components:
at least one measuring sensor on the person or the animal for the acquisition or the monitoring of medically relevant data, such as in particular data, which describe the cardio vascular and pulmonary function and/or contained data regarding blood values or blood composition, which sensor comprises at least one light source which can emit light at least at two wavelengths, as well as at least one light receiver for determining the light transmitted through a tissue portion of the person, and
at least one light source frequency modulating means to frequency modulate the optical radiation of the light source at a carrier frequency in order to shift the power spectrum of the pulsoximeter signals. The basic idea of using AC-Coupling or Lock-In Amplification (synchronous detection), is to temporarily modulate the amplitude of the optical radiation of, e.g., the LED at a carrier frequency f_{c} in order to shift the power spectrum of the pulsoximeter signals into a higher frequency range where environmental optical radiation is unlikely and electronic band pass filtering is technologically less stringent. Thus, the pulsoximeter signals are readily discriminated from electronic and parasitic contributions of environmental optical radiation outside the frequency range of, e.g. f_{c} +/−5 Hz, increasing significantly the S/N (Signal/Noise)- and S/B ratio.

Further specific designs of the configurations are described within the dependent claims.

Furthermore, the above mentioned problem is solved according to the invention by means of methods according to the invention. Proposed is a method for monitoring e.g. pulsation frequency, oxygen saturation in blood or breathing frequency, which comprises at least one of the following steps:
measuring or monitoring medically relevant data of a person or an animal, such as in particular data, which describe the cardiovascular and pulmonary function and/or contain data regarding blood values or blood composition with the use of at least one measuring sensor, which sensor comprises at least one light source which can emit light at least at two wavelengths:
direct the emitted light or optical radiation, respectively, by using a beam shaping element, such as e.g. a diffractive or refractive lens to the human or animal tissue;
receiving and detecting the emitted and shaped light with at least one light receiving element for determining the light transmitted through the tissue portion of the person or the animal.

In addition to the mentioned method or as alternative, it is further proposed to filter the emitted light by using geometrical baffles or light traps, respectively, and/or optical wavelength filters to suppress by geometric and/or optical means the parasitic contribution of environmental radiation.

Again, in addition to the above mentioned two methods or as an alternative, it is further proposed to temporarily modulate the amplitude of the optical radiation of the light source by using e.g. AC-Coupling or Lock-In Amplification detection means. The basic idea of using AC-Coupling or Lock-In Amplification detection means is to temporarily modulate the optical radiation of, e.g., the LED at the carrier frequency f_{c} in order to shift the power spectrum of the pulsoximeter signals into a higher frequency range where an environmental optical radiation is unlikely and electronic band pass filtering is technologically less stringent.

Further preferred methods are described in the dependent claims.

Further preferred embodiment variants, in particular of an ear sensor employed for measurements by means of pulsoximeter, are found in the international patent application WO 02/089 663 which herewith is included as an integral component of the present patent application.

The invention will be explained in further detail by examples and with reference to the enclosed figures.

Therein depicted:

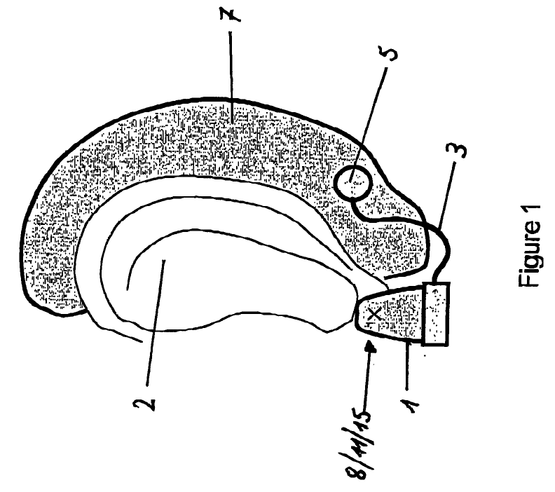

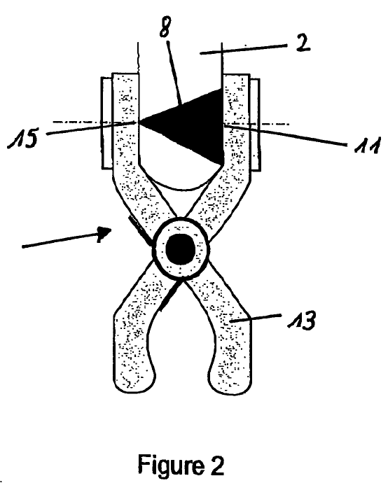

FIG. 1 schematically the arrangement of an ear clip for oximetric measurement;

FIG. 2 schematically the ear clip of FIG. 1 in cross section view;

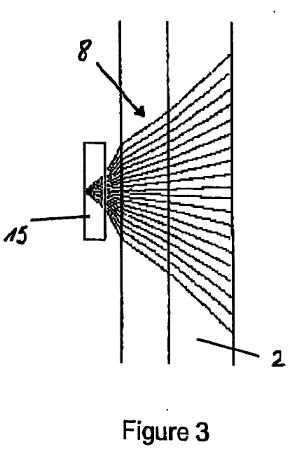

FIG. 3 schematically a light source to be used in an oximetric sensor without beam shaping optics;

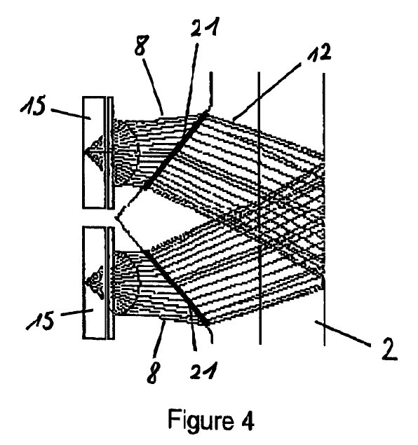

FIG. 4 schematically two light emitting sources for an oximetric sensor including beam shaping optics;

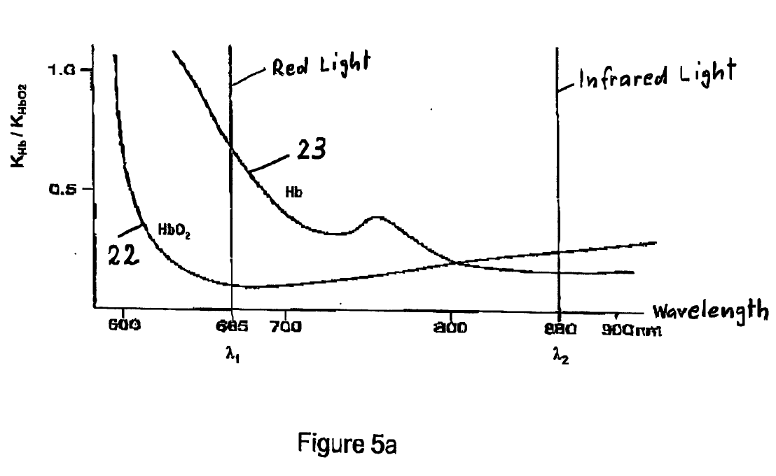

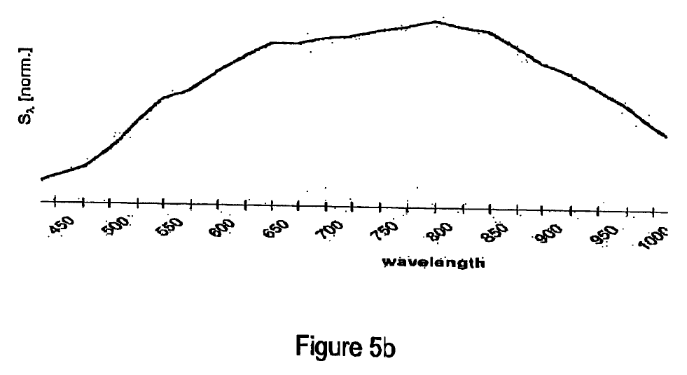

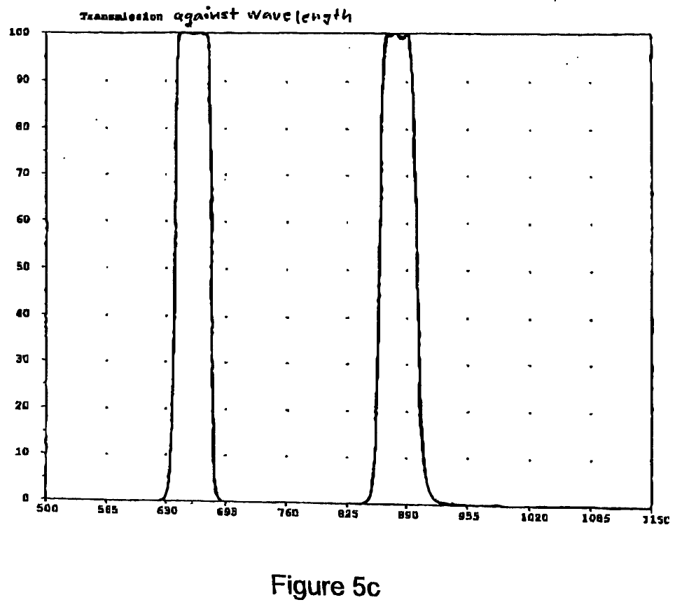

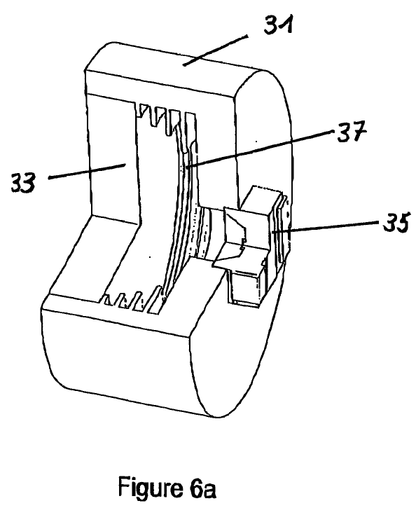

FIG. 5a a diagram showing the light absorption curves of with oxygen saturated (HbO₂) and unsaturated (Hb) haemoglobin;

FIG. 5b a diagram showing the spectrum sensitivity of a photo detecting element;

FIG. 5c in a diagram the transmission spectrum of a double band pass filter;

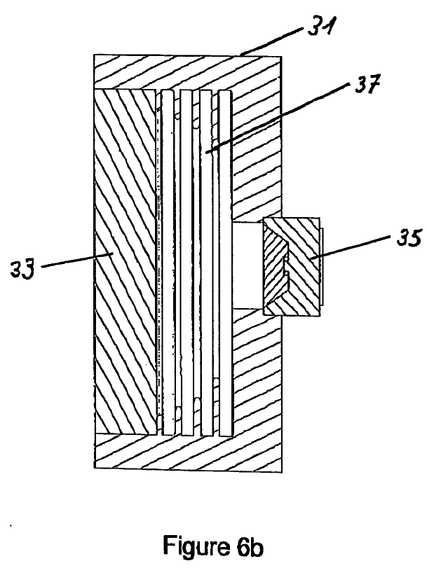

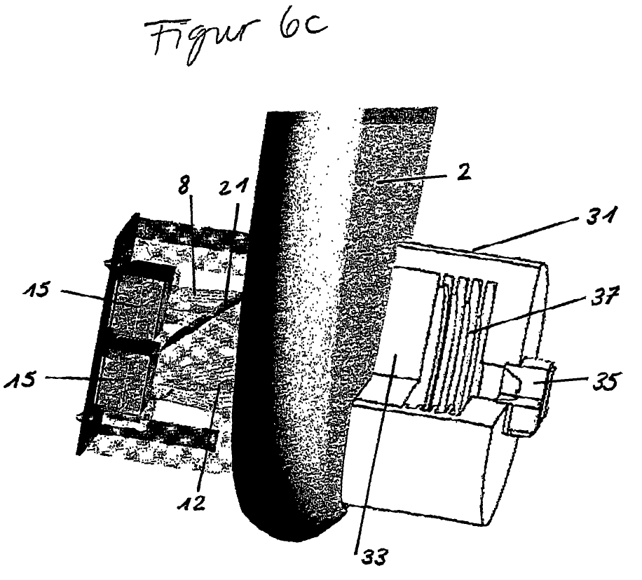

FIG. 6a in perspective view a part of an oximetric sensor with arranged baffles to avoid stray light;

FIG. 6b the part of the sensor of FIG. 6a in longitudinal section;

FIG. 6c an oximetric sensor in perspective view, containing optical lenses, filters and geometrical baffles;

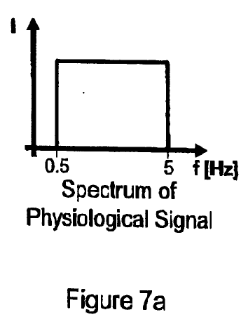

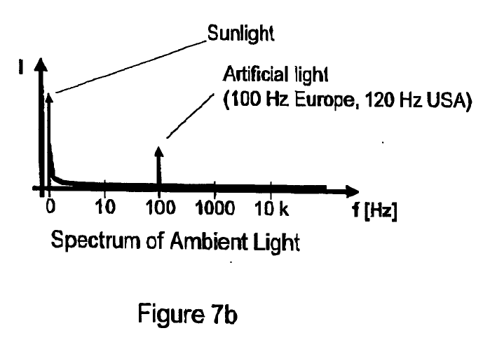

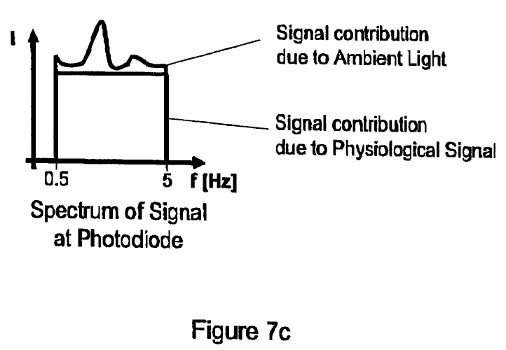

FIG. 7a a diagram showing power spectrum of physiological signals;

FIG. 7b a diagram showing power spectrum of ambient light;

FIG. 7c a diagram showing power spectrum of physiological signals and ambient light without phase shifting or modulation of the light source of a sensor,

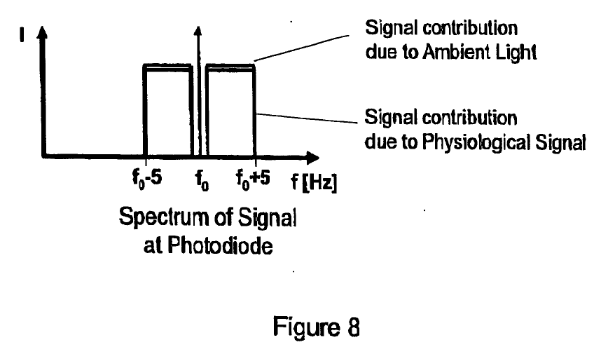

FIG. 8 a diagram showing power spectrum of physiological signals and ambient light with phase shifting or modulation of the light source of a sensor;

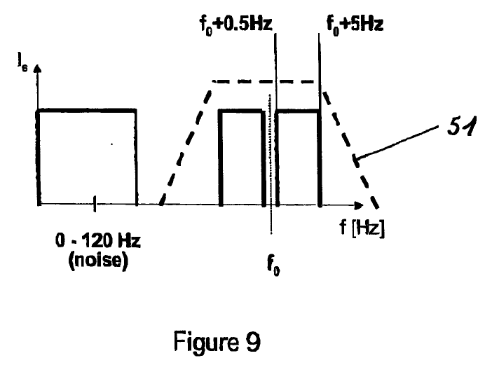

FIG. 9 a principal of using band pass filtering means at a sensor with applied phase shifting or modulation of the light source at a sensor, and

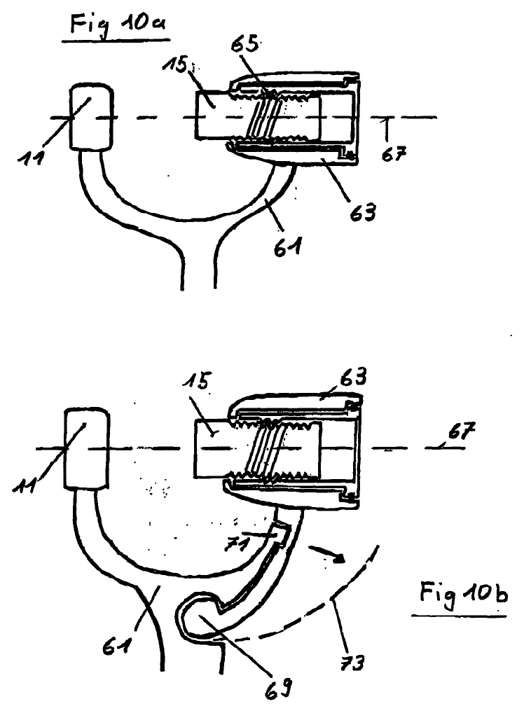

FIGS. 10a+b a further fixing system for arranging a pulsoximetric sensor system as an alternative to a clip according to FIGS. 1 and 2.

FIG. 1 shows schematically the arrangement of an ear sensor 1 which can be arranged in form of an ear clip. This sensor 1 can be arranged e.g. at an earlobe of ear 2. Furthermore, the sensor or ear clip is connected via a wire 3 and the connection 5 with the main unit 7 including e.g. a power source, like a battery, and measuring and/or monitoring electronics.

In FIG. 2, the ear clip 1 is shown in cross section where it can specifically be seen that the sensor is designed in form on a clip 13. The sensor or ear clip 13 furthermore includes a light source 15 which emits a light beam 8 to a light receiver 11. The light is guided or emitted through the ear skin or earlobe 2.

As already mentioned in the introduction, the sensor is working according to the oximetric principal which is known best out of the state of the art. Optical pulsoximetry is used for non-invasive measurement, e.g. for pulsation and oxygen saturation in the human body. The light source is emitting light at two wavelengths, at 660 nm and a second wavelength within the range of 800 to 1000 nm, which means in the present case at 890 nm. Therefore, it is of course also possible to have two light emitting sources arranged, which means two LEDs. The light receiver is determining the light transmitted through the earlobe, which means through the tissue portion of a person to be surveyed.

Within the main unit 7 the measured values can be compared with reference values being representative for a certain health status of the person to be surveyed.

Of course, the sensor can also be arranged at other parts of the human body, such as e.g. at a finger or a toe. In addition, the monitoring can also be executed at animals, which means that pulsoximetric sensors can also be arranged e.g. at the ear of animals, such as e.g. cows. According to an alternative design of the sensor, it could also be possible to arrange the light receiver in such a way so that the light reflected through the earlobe is determined. Again, according to a further alternative, it could even be possible by arranging at least two light receivers to determine the light transmitted through the earlobe and the light reflected by the earlobe.

FIG. 3 shows a light beam emitted by a LED where it is clear that most of a light beam with such a large spreading angle does not hit the receiver. Walking around through various rooms, one time halogen light is influencing the light beam 8, the other time conventional light is influencing the measurement, and again at another time, for the person using a car, the sunlight is influencing the measurement, e.g. if sunlight and shadow alternate within a short period of time.

Therefore, it is proposed, as shown in FIG. 4, to use beam shaping optics 20 to direct the emitted optical radiation 8 emitted from the two LEDs 15 to the middle of the earlobe. As it is shown clearly in FIG. 4, using the beam shaping optics 21, the two initial light beams 8 are guided in form of bundled beams 12 to a relatively small area within the middle ear 2. By using the beam shaping optics 21, of course the influence of environmental light or noise, respectively, can be reduced substantially by increasing the S/B ratio. First or all, the light beam is bundled and, in addition, the optical signal power can be increased.

As an alternative or in addition to using beam shaping optics, it is also possible to influence the sensor architecture of the pulsoximetric sensor. First of all, it is possible to use a light receiving or light sensitive element 11 with reduced light sensitivity outside the spectral range of the band limited light source as LEDs. FIG. 5a shows the light absorption curves of with oxygen saturated 22 and unsaturated 23 blood. As visible from the shown diagram, the sensor architecture, which means the spectrum sensitivity, should be in the range within approximately 500 nm to approximately 1000 nm. In addition, in FIG. 5a the two wavelengths λ₁ and λ₂ are indicated at which the pulsoximetric sensor is operated.

As a consequence, FIG. 5b shows the spectrum sensitivity of a silicon photo detecting element which is suitable for the use in a pulsoximetric sensor according to the present invention. As shown, the detection sensitivity is within a range of approximately 500 to 1000 nm. In other words, any light below or above this range would not be detected by the light receiving element with a sensitivity as shown in FIG. 5b. In addition, it is possible to arrange an optical wavelength filter or double pass filter which is e.g. light permeable at the wavelength of approximately 660 nm and in the range of approximately 850 nm to 910 nm. A corresponding transmission spectrum of such a double band pass filter will be suitably used in a pulsoximetric sensor as shown in FIG. 5c.

Preferably, the two means, as described with reference to FIGS. 5b and c, are combined as wavelength filters might be also light permeable in lower wavelengths areas and higher wavelengths areas which, by using a selective light detecting element, can be eliminated.

Of course, it is furthermore possible to combine wavelength filters, wavelength sensitive receivers like photodiodes, with beam shaping optics as described with reference to FIG. 4.

A further possibility for the better performance of a pulsoximetric sensor, is to arrange geometric means as e.g. so-called geometrical baffles (light trap). In FIG. 6a, a part of a pulsoximetric sensor is shown, which means the part of the sensor after the transmitted light has passed, e.g. the earlobe of a human or animal individual. Within the mentioned sensor part 31, after e.g. a double pass filter 33, circumferential extending baffles 37 are arranged to avoid stray light to reach the photo detecting element.

For any stray light which has entered the sensor e.g. before or at the area of the earlobe, will be trapped within the depressions of the baffles 37, and therefore will not substantially influence the emitted light of the LEDs.

FIG. 6b shows the part of the sensor of FIG. 6a in a longitudinal section. The stray light will be trapped substantially within the depressions of the baffles 31, while the emitted light by the LEDs will reach the optical sensor 35.

According to the preferred embodiment of the invention, the various described optical and geometric means, such as the beam shaping element as shown in FIG. 4, the wavelength filters, the sensor architecture, and the mentioned baffles, can be combined as shown in principle and perspective view in FIG. 6c. Again, light is emitted from the two LEDs 15 and is shaped by the two beam shaping elements or lenses 21 to be guided as beams 12 through the earlobe 2. After the earlobe, the double pass filter 33 is arranged to guarantee that only light in the range of approximately 660 nm and in the range of approximately 890 nm is transmitted through the filter. After the filter, any stray light, entered the sensor e.g. trough the earlobe from the side, will be trapped within the baffles 37 which are arranged in circumferential direction. Finally, a photo detecting element 35 is arranged with specific spectrum sensitivity.

By using sensor architecture as shown in FIG. 6c, the Signal-to-Background ratio may be increased in a range of a factor 50 to 1000.

According to a further aspect of the present invention, it is furthermore possible to use a light source modulation to temporarily modulate the optical radiation of the LED.

The basic idea of using AC-Coupling or Lock-In Amplification (synchronous detection), is to temporarily modulate the optical radiation of the LED at the carrier frequency f_{c} in order to shift the power spectrum of the pulsoximeter signals into a higher frequency range where environmental optical radiation is unlikely and electronic band pass filtering is technologically less stringent. AC-Coupling or Lock-In Amplification is well known out of the state of the art and is described in literature 3.

FIG. 7a shows a spectrum of physiological signals, such as pulsation frequency, breathing frequency, etc. The frequency of physiological events is within the range of approximately 0.5 Hz (30 heartbeats in one minute) up to approximately 3 Hz (180 heartbeats in one minute) that can be even higher and therefore is supposed to go up to 5 Hz.

The frequency spectrum of ambient light is schematically shown in diagram 7b. Sunlight is at 0 Hz, while artificial light, such as e.g. electrical in-house light, is going up to approximately 120 Hz (USA). In other words, within the range of frequencies of physiological signals, we have high influence of frequencies of sunlight and ambient light. A corresponding combined frequency spectrum is shown in Fig. c, which would be detected by a photo diode without the use of any means as described above in relation to FIGS. 1 to 6. FIG. 7c shows a basic signal contribution due to physiological signal and additional signal contribution due to ambient light. In other words, the influence of ambient light is quite substantial, and therefore the deviations of the measured values compared to the real values can be dramatic.

Besides the high influence of ambient light, also sunlight can have a dramatic influence, e.g. if a person is walking through streets with relatively quick changing conditions between sunlight and shadow. Another serious possibility is caused by a tree avenue when driving along the trees. Sunlight then is received e.g. by the pulsoximetric sensor at a certain frequency, which means that every time when passing a tree, sunlight is attenuated and between the trees sunlight is influencing the measurement of the pulsoximetric sensor.

As a consequence, it is therefore proposed to emit light by the LEDs not as current or continuous light but as pulsed light. The frequency is chosen in such a way that it is outside the frequency spectrum of sunlight and of ambient light which, according to FIG. 7b, is in the range of above approximately 1000 Hz. Thus, the pulsoximeter signals are readily discriminated from electronic and parasitic contributions of environmental optical radiation outside the frequency f_{c}+/−5 Hz increasing significantly the Signal-to-Noise and Signal-to-Background ratio. FIG. 8 shows the shift spectrum of signal to a region where there is little influence, e.g. of ambient light. F₀ is the chosen frequency of the emitted light to operate the pulsoximeter sensor and the range between f₀−5 Hz and f₀₊₅ Hz is the consequence of the influence of the frequency due to physiological signal. Therefore, as shown in FIG. 8, the frequency spectrum of signal at the photo diode does have a basic signal contribution due to physiological signal. The signal contribution which is shown at the top of the signal contribution due to physiological signal and which is due to ambient light, is very small and as a consequence is approximately neglectable. Any noise or sunlight within the range of 0 to 120 Hz, while the light beam for the pulsoximetric measurement is within the range of approximately f₀−5 Hz to f₀+5 Hz, will not influence the measurement of the pulsoximetric sensor. F₀ could be e.g., as mentioned, 1000 Hz which of course is a frequency far outside of any indoor light source, as e.g. halogen light, conventional light, etc. f₀ of course can be chosen at any other frequency, as e.g. 2000 Hz or even higher. By using light source modulation, it is even possible to use an additional filter removing a certain frequency spectrum. Looking e.g. at FIG. 9, it is possible to arrange a filter band pass 51 which is e.g. removing any frequencies in the range of 0 to 120 Hz. The respective filter is shown in form of the dashed line 51. As a result, we end up by a diagram according to FIG. 9b only showing any measurements in the range of f₀−5 Hz to f₀+5 Hz.

Finally, after the measurements with pulse light have been executed, of course a reversed phase shifting or modulation has to be executed to calculate the real values of the Pulsoximetric measurement. Again, this reverse face shifting on modulation according to Lock-In technique is known out of the state of the art.

Again, it is of course possible to combine the light source modulation as described with reference to FIGS. 8 and 9 with any of the prior means such as the sensor architecture, as shown with respect to FIGS. 5 and 6 and with beam shaping optics, as described in FIG. 4.

By using one of the proposed devices or methods, respectively, according to the present invention or a combination thereof, it is possible to use pulsoximetric measurement or monitoring to survey the health condition of a person or an animal which is mobile. In other words, pulsoximetric measurement is not restricted for use in, e.g., a hospital but can also be used, if a person is travelling, is staying at home, etc. Furthermore, it is also possible to study health conditions of animals living in nature such as e.g. cows feeding outside.

Coming back to the fixing system, which means a clip as shown in FIGS. 1 and 2, it has to be mentioned that when using a clip for fixing a pulsoximetric sensor, problems could occur due to strong movements of the human or animal individual or due to swelling or contracting of the human or animal tissue during the measurement with the pulsoximetric sensor. In other words, if e.g. an earlobe of an ear 2, as shown in FIG. 2, would swell, than the distance between the LED 15 and the photo detector 11 would increase and, what is even more critical, the beam path could divert substantially from the optical axis of the LED and the photo detector. Therefore, it is preferred to further provide means for stabilizing the signal guiding and detecting and to provide means for the beam path to be co-linear with the optical axis of the LED and the photo detector. Because of that, according to FIGS. 10a and 10b, it is proposed to use a frame 61 which is stable and does not change its dimensions due to strong movements or an individual carrying the pulsoximetric sensor or due to swelling or contracting of the tissue to be monitored by the pulsoximetric sensor. In this case, of course, other means have to be provided, so that the distance between the LED 15 and the photo detector 11 can be adjusted or adapted to the thickness of the tissue to be monitored. Therefore, according to FIG. 10a, it is proposed that the LED 15 is arranged within a clamping mechanism 63 and that between the clamping mechanism 63 and the LED a screw connection 65 is arranged, so that the LED 15 can be moved into the clamping mechanism or out of the clamping mechanism 63. In other words, the distance between the LED 15 and the photo detector 11 can be adjusted along the optical axis 67 which guarantees that the beam path has always been co-linear with the optical axis 67 of the LED and the photo detector.

Comparing the clip mechanism according to FIGS. 1 and 2 and the frame 61 as shown in FIG. 10a, it is obvious that in using a frame it is not easy to arrange or remove the pulsoximetric sensor to or from an earlobe of an ear, if required, e.g. if a person wearing the pulsoximetric sensor is taking a bath, a shower, etc. Therefore, it is proposed, as shown schematically in FIG. 10b, to use a snap-in mechanism 71, which means that the clamp mechanism 63 holding the LED 15 can be rotated e.g. in direction ot dashed line 73 around an axis 69 and removed from the frame 61 or vice versa can be arranged at the frame 61 by arranging within the axis 69 and within the snap mechanism 71. Therefore, the LSD 15 has not to be rotated within the screw connection 65 between the clamp mechanism 63 for removing the frame 61 from an earlobe of an ear.

The invention, as described with reference to FIG. 1 to 10, is of course not limited to the examples as shown in FIG. 1 to 9, but can be differently designed combined with other features, etc. E.g. pulsoximetric measurements can also be done at other parts of the body like e.g. fingers or toes. In addition, not only one light source can be used for the measurement, but also two or even more light emitting sources. It is understood that also one, two, or more light receiving detectors can be used. All the various above mentioned and proposed means for improving the pulsoximetric monitoring to survey the health condition are not restricted to the measurement of transmitted light through a human or animal tissue. All the proposed means according to the present invention can be used, of course, also by measuring reflected light or as a combination of measuring reflected and transmitted light through a human or animal tissue.

Furthermore, all the above mentioned means for improving the measurement of the oxygen saturation of blood using a light source can, of course, also be used by any further kind of measurements using a light source such as, e.g., non-invasive monitoring or arterial carbon dioxide partial tension, the content of blood sugar, etc. In other words, for any kind of measuring blood properties using light emission through a human or animal tissue, the above mentioned means for improving the measurement can be used. This means that the present invention is not at all restricted to optical pulsoximetry used for non-invasive measure of pulsation and oxygen saturation in arterial human or animal blood.

The measured values can be transmitted via a wire connection or wireless, e.g. within the range of radio frequency. Well known these days is wireless transmission using “Bluetooth” technology. According to a further embodiment, the pulsoximetric sensor could be included within a hearing aid device.

Taking prior art into consideration, the measured values can be monitored at a special unit worn by the person or patient, respectively, where e.g. a signal is generated, if the measured value is not within a predetermined range. In other words, health problems could be detected and an alarm signal could be generated which can be transmitted to a respective person, to a medical doctor, to a hospital, etc. so that help can be organised. Furthermore, it is possible to include e.g. a so-called GPS device which at any time gives the location of the person using the pulsoximetric sensor monitoring configuration.

1. Configuration for the acquisition and/or monitoring of medical data, in particular the state of the cardiovascular and pulmonary system, blood values or blood composition, characterised by at least one measuring sensor for the acquisition of the medical data such as the stare of the cardiovascular system, etc. of a person comprising at least one light source which can emit light at least at two wavelengths, as well as at least one light receiver for determining the light transmitted and/or reflected through a tissue portion of a person or an animal further comprising means in order to increase the optical Signal-to-Noise and/or Signal-to-Background ratio.

2. Configuration according to claim 1 and at least one beam shaping optical element to direct the emitted light into a human or animal tissue and the light receiver.

3. Configuration according to claim 2, characterised in that the beam shaping element is a diffractive or refractive beam shaping element.

4. Configuration according to one of the claims 1 to 3, characterised in that at least two light emitting sources, such as LEDs, are arranged and that two beam shaping elements are arranged to direct the emitted light into the same area within the human or animal tissue and that the light receiving element is a photo detecting element.

5. Configuration for the acquisition and/or monitoring of medical data, in particular the state of the cardiovascular and pulmonary system, blood values or blood composition, characterised by at least one measuring sensor for the acquisition of the medical data such as the state of the cardiovascular system, etc. of a person comprising at least one light source which can emit light at least at two wavelengths, as well as at least one light receiver for determining the light transmitted and/or reflected through a tissue portion of a person or an animal and at least one light tray and/or optical wavelength filter.

6. Configuration according to claim 5, characterised in that the optical wavelength filter is an optical double band pas filter.

7. Configuration according to claim 5, characterised in that the light receiver has such a limited detection sensitivity that the two frequencies of the light source are within the sensitivity area of the receiver.

8. Configuration according to claims 1 to 7, characterised in that at least a wavelength filter and/or a light trap, such as geometrical baffles, are adapted to suppress, by geometric and/or optical means, the parasitic contribution of environmental radiation in order to increase and stabilise the signal/background ratio versus environmental conditions.

9. Configuration for the acquisition and/or monitoring of medical data, in particular the state of the cardiovascular and pulmonary system, blood values or blood composition, etc., characterised by at least one measuring sensor for the acquisition of the medical data, such as the state of the cardiovascular and pulmonary system, etc. of a person comprising at least one light source which can emit light at least at two wavelengths, as well as at least one light receiver for determining the light transmitted and/or reflected through a tissue portion of a person or an animal,
at least one beam shaping optical element to direct the emitted light into a human or animal tissue and the light receiver, and
at least one light trap such as geometrical baffles and/or an optical wavelength filter, such as a double band pass filter.

10. Configuration according to claims 1 to 9, comprising light source amplitude modulating or light source modulating means to shift the frequency of the emitted light.

11. Configuration according to claim 10, comprising a light source amplitude modulating means to modulate the frequency of the emitted light in a frequency range substantially outside of frequency of noise and/or environmental signals.

12. Configuration according to claim 10 or 11, comprising means for light source amplitude modulation or light source modulating means to shift the frequency of the emitted light in a range where environmental disturbances are substantially neglectable.

13. Configuration according to one of the claims 10 to 12, comprising means for light source amplitude modulating or light source modulating means to shift the frequency of the emitted light in a range of above 120 Hz, preferably above 500 Hz.

14. Configuration according to one of the claims 1 to 13, comprising mechanical fixing means for arranging the configuration at a human or animal tissue as e.g. at an earlobe of an ear, the means guaranteeing that the beam path between the light emitter and the light receiver is always co-linear with the optical axis of the light emitter and the light receiver.

15. Configuration according to claim 14, wherein the means for fixing include a rigid frame with two U- or V-like arranged arms, where in the area of the one arm end the photo detector is arranged, and at the area of the other arm end a clamping mechanism within the LED is arranged screwably connected to the clamping mechanism, so that the distance between the light receiver and the light transmitter can be varied in such a way that the beam path between the light emitter and light receiver always is co-linear with the optical axis of the light emitter and light receiver.

16. Configuration according to claim 15, wherein the arm of the frame wearing the clamp mechanism with the light emitter is removably attached to the frame, the connection between the frame and the removable arm being a snap-like mechanism to ensure that the removable arm is fixed to the frame in a constant, predetermined manner.

17. Pulsoximetric sensor, including a configuration according to one of the claims 1 to 16.

18. Method for measuring and/or monitoring of medical data, in particular the state of the cardiovascular and pulmonary system, blood values or blood composition, etc.,
